(19) United States
(12) Patent Application Publication
(10) Pub. No.: US 2008/0234722 A1
(43) Pub. Date: Sep. 25, 2008

(54) Inferior vena cava filter on guidewire

(75) Inventors: Michael J. Bonnette, Minneapolis, MN (US); Eric J. Thor, Arden Hills, MN (US); Daniel T. Janse, Lino Lakes, MN (US); Ernest R. Scherger, Center City, MN (US)

Correspondence Address: GREGORY L BRADLEY;MEDRAD INC, ONE MEDRAD DRIVE, INDIANOLA, PA, 15051, US

(73) Assignee: Possis Medical, Inc.

(21) Appl. No.: 11/811,974

(22) Filed: Jun. 13, 2007

Related U.S. Application Data
(60) Provisional application No. 60/813,570, filed on Jun. 14, 2006.

(51) Int. Cl. A61F 2/01

(52) U.S. Cl. 606/200

(57) ABSTRACT

A temporary inferior vena cava filter including a guidewire and a doublet cage filter distally located on the guidewire. The doublet cage filter has a proximal cage filter and a distal cage filter, both of resilient and biased toward their expanded or deployed state. The proximal and distal cage filters may be collapsed by actuation, preferably with a sheath. A method of protecting from pulmonary embolism during treatment of a deep vein thrombosis is disclosed. The doublet cage provides stability when deployed in the inferior vena cava, is readily retrieved and readily manufactured. A method of manufacturing the doublet cage filter assembly is also disclosed and involves a nitinol tube with plural cuts to form struts which are heat treated in an expanded state.

### CROSS REFERENCES TO RELATED APPLICATIONS

This application claims benefit from the earlier filed U.S. Provisional Application No. 60/813,570 entitled “Catheter” filed Jun. 14, 2006, and is hereby incorporated into this application by reference as if fully set forth herein.

### BACKGROUND OF THE INVENTION

1. Field of the Invention

The present invention is for a filter on a guidewire and, in particular, relates to a peripheral guidewire with a deployable doublet filter for temporarily protecting the inferior vena cava of a patient from passage of thrombus debris leading to pulmonary embolism during treatment for deep vein thrombosis.

2. Description of the Prior Art

Deep vein thrombosis (DVT) is a dangerous medical condition in which a blood clot forms in a large vein, typically a large vein in a leg. This condition is also more commonly known as “traveler's thrombosis” or “economy-class syndrome” and is believed to be particularly associated with sitting motionless for long periods of time. Consider, for example, a vein in the leg which becomes burdened with a large blood clot. The leg then becomes quite painful and swollen and may eventually even develop open sores. If all or a portion of the clot is liberated from the original site in the leg, such debris will travel through the vein toward the heart, traveling in particular through the inferior vena cava (IVC) and then into the heart for subsequent pumping to the lungs. Next, such liberated debris lodges in the vasculature of the lungs, generating far more serious medical consequences for the patient. This result is known as a pulmonary embolism (PE). Pulmonary embolism is a blockage of the vasculature of the lung, and can destroy the affected lung tissue, as well as its normal function. It has been estimated that if left untreated, roughly one-in-three pulmonary embolisms will prove fatal, and also that between one-in-twenty or one-in-ten pulmonary embolisms are fatal within the first hour of occurrence. Therefore, interventional medical strategies are often employed to eliminate the thrombosis or clot while still located in the vein of the leg. Such is the significance of the earlier mentioned deep vein thrombosis or DVT.

The interventional strategies for addressing deep vein thrombosis are varied. Historically, heparin treatment has been employed, but heparin treatment tends to leave the deep vein thrombosis in place and serves more to prevent the formation of new sites of deep vein thrombosis while also reducing the occurrence of pulmonary embolism. The patient, however, continues to be plagued by swelling, pain, and possibly eventual open sores on the leg. More aggressive interventional strategies include application of fibrinolytic agents, more commonly called “clot buster drugs,” which begin to break down and/or dissolve the clot, and thrombectomy operations which aim to physically cut up and remove the clot. Significantly, these more aggressive interventional strategies may be accompanied by an increasing danger of inadvertently liberating chunks of debris, again leading to increased possibility of pulmonary embolism. Note that if a liberated chuck of debris or clot passing through the inferior vena cava is sufficiently large to inhibit pulmonary function, then the event is classified as a pulmonary embolism.

To address this possible inadvertent liberation of chunks of debris during aggressive intervention while eliminating the deep vein thrombosis, a filter mechanism is at times employed between the site of the deep vein thrombosis being treated and the heart. In particular, the filter typically is located within the inferior vena cava so as to capture and thereby prevent passage of larger liberated chucks of debris into the heart and then on to the lungs. Such filter mechanisms are termed inferior vena cava filters or “IVC filters.” The currently available IVC filters may be permanent or temporary installations. Unfortunately, currently available IVC filter mechanisms are also plagued by shortcomings. In particular, currently employed IVC filter mechanisms may spontaneously generate a clot or thrombosis centered at the IVC filter. Further, because the IVC filters are generally temporarily placed by penetrating fine projecting hooks through an inside wall of the inferior vena cava, the hooks attachments to the inside wall may fail and allow inadvertent migration of the IVC filter toward the heart, or the hooks may fully puncture the wall of the inferior vena cava, or if a temporary IVC filter is left in a patient too long, it may unintentionally become a virtually permanent IVC filter. Additionally, removing a temporary IVC filter involves snaring the temporary IVC filter and pulling it free from the inferior vena cava interior wall. In other words, removal and retrieval of the temporarily implanted IVC filter can be an unexpectedly complex operation, fraught with additional undesirable complications. Given these many shortcomings and challenges, some physicians view the risk associated with temporarily implantable IVC filters as too extreme and proceed to aggressively intervene in treating a deep vein thrombosis without employing any protective IVC filter.

Clearly there is a need for a new IVC filter which may be temporarily deployed during aggressive interventional treatment of a deep vein thrombosis. Such a new IVC filter would provide the advantages of filtration and avoid the many shortcomings of the current temporary implantable IVC filters. The present invention, as explained below, is a device which answers this need. It is easily deployed and easily retrieved. Further, it is readily manufactured. Most importantly, it provides protection from pulmonary embolism while performing aggressive interventional treatment of deep vein thrombosis.

### SUMMARY OF THE INVENTION

The general purpose of the present invention is to provide a filter for protection against pulmonary embolism during aggressive intervention treatments for deep vein thrombosis.

According to one embodiment of the present invention, there is provided a temporary inferior vena cava filter. The temporary inferior vena cava filter includes a guidewire and a two-cage or doublet cage filter. The guidewire has a distal and a proximal end. The two-cage or doublet cage filter is distally situated on the guidewire and includes a first resilient filter cage and a second resilient filter cage. Both the first and second resilient filter cages are actuatable between a collapsed state and a deployed state. The second resilient filter cage is situated distal to the first resilient filter cage. Preferably, the first resilient filter cage and the second resilient filter cage of the two-cage filter are actuated between the collapsed state and the deployed state by a sheath. More preferably, the sheath is of a polyimide material. Most preferably, the sheath is braided polyimide with a size of about # 6 or # 7 French. The doublet cage assembly is preferably formed of nitinol. More preferably, nitinol tubing receives a first plurality of parallel cuts in the nitinol tubing at the desired location for one of the doublet cages and a second plurality of parallel cuts for the other of the doublet cages. The cuts define struts. The cuts of a particular plurality of cuts can be oriented linearly (i.e., longitudinally and parallel to the tube axis) or helical relative to the tube axis. The struts are then heat set in an expanded deployed state to provide the resilient expanded characteristic to the filter cage. Advancing a sheath over the filter cages forces the two-cage filter to a collapsed state and retracting the sheath allows the two-cage filter to resiliently expand to the expanded or deployed state. In the deployed state, each filter cage has a convex exterior. Preferably, there are from about eight to about 16 cuts and, therefore, about eight to 16 struts in the pluralities of cuts in the nitinol tube. If helical, the cuts are oriented at about a 25 degree angle.

In another embodiment, the present invention is a method of providing temporary protective filtering to protect from pulmonary embolism for a patient being treated for deep vein thrombosis. In the method are included steps of providing a guidewire with a doublet cage filter, as described above; inserting the two-cage filters on the guidewire into a vein of the patient and advancing the two-cage filters to a protective location distally relative to the deep vein thrombosis, and then deploying the two-cage filters at the protective location; treating the deep vein thrombosis; retracting the two-cage filters to the collapsed state by advancing a sheath; and withdrawing the two-cage filters in the collapsed state with the attached guidewire and sheath. The method also may include debulking the two-cage filter prior to retraction within the sheath.

In still another embodiment, the present invention is a method of forming or manufacturing a filter cage assembly for attachment to the distal end of a guidewire. The method includes the steps of providing a tube; and cutting a plurality of parallel cuts in the tube to define a plurality of struts. The tube is nitinol and the cuts are parallel and may be linear or helical. If the cuts are helical, then they are oriented at about 25 degrees to the tube axis. The struts are expanded, preferably by a heat resistant insert, and then heat treated.

One significant aspect and feature of the present invention is the continuous attachment of the new IVC filter to a peripheral guidewire.

Another significant aspect and feature of the present invention is the lack of hooks on the new IVC filter.

Still another significant aspect and feature of the present invention is the ease of initial deployment of the new IVC filter in a patient.

Yet another significant aspect and feature of the present invention is the ease of subsequent removal of the new IVC filter from a patient.

Yet another significant aspect and feature of the present invention is the ease of manufacture of the new IVC filter.

Yet another significant aspect and feature of the present invention is the improved wall apposition within the inferior vena cava of a patient.

Yet another significant aspect and feature of the present invention is that thrombotic debris that is located internally in either of the cages will likely be macerated as the doublet is withdrawn into the sheath.

Having thus described embodiments of the present invention and set forth significant aspects and features of the present invention, it is the principal object of the present invention to provide an IVC filter for protection from pulmonary embolism in a patient being aggressively treated for deep vein thrombosis.

### BRIEF DESCRIPTION OF THE DRAWINGS

Other objects of the present invention and many of the attendant advantages of the present invention will be readily appreciated as the same becomes better understood by reference to the following detailed description when considered in connection with the accompanying drawings, in which like reference numerals designate like parts throughout the figures thereof and wherein:

FIG. 1 is a temporary IVC filter, shown in an expanded or deployed state with an associated sheath retracted, the present invention;

FIG. 2 is the temporary IVC filter of FIG. 1, shown in a contracted or undeployed state with the associated sheath extended or advanced to cover the doublet filter (portions of the sheath are removed to allow the underlying doublet filter to be viewed);

FIG. 3 is the expanded doublet filter cage assembly (prior to joining to guidewire subsequent to heat treatment);

FIG. 4 is the doublet filter cage assembly, collapsed, prior to expansion and subsequent to forming cuts therein;

FIG. 5 is an alternative embodiment of the doublet filter cage assembly showing its expanded or deployed state, in an isometric view;

FIG. 6 is the alternative embodiment of the doublet filter cage assembly of FIG. 5 in a contracted state subsequent to cutting and prior to expanding and heat treating; and,

FIG. 7 is an exemplary schematic view of a method of use of the present invention.

### DETAILED DESCRIPTION OF THE PREFERRED EMBODIMENTS

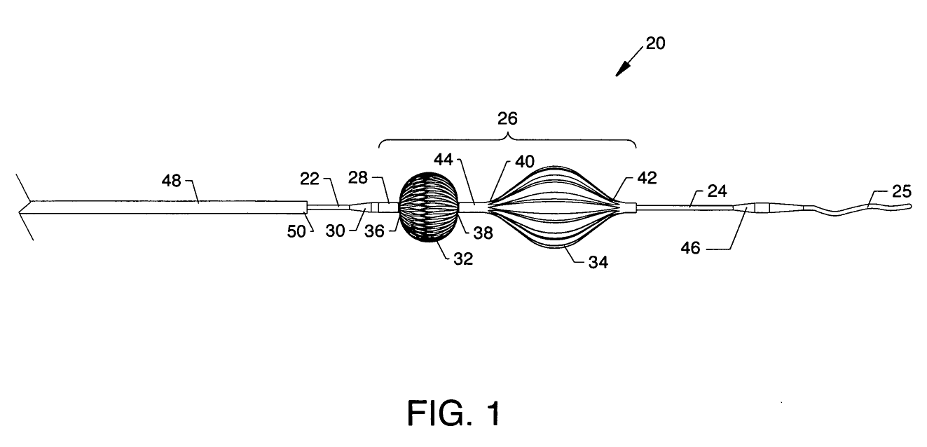

FIG. 1 shows a temporary IVC filter 20, the present invention. The temporary IVC filter 20 includes a guidewire 22. The guidewire 22 has a proximal end (not shown) and a distal end 24. The distal end 24 includes a shapeable tip 25. A doublet filter cage assembly 26 overlies the guidewire 22 adjacent the distal end 24 and has a connection 28 to the guidewire 22. The connection 28 is proximally located on the doublet filter cage assembly 26 and generally distally located on the guidewire 22. A proximal cone 30 is present at or adjacent to the connection 28 and is directed proximally (i.e., proximal cone 30 has a smaller diameter oriented proximally and a greater diameter oriented distally). The doublet filter cage assembly 26 includes a proximal cage 32 and a distal cage 34. The proximal cage 32 has a proximal end 36 adjacent the connection 28 and a distal end 38 located distal to the connection 28. The distal cage 34 has a proximal end 40 and a distal end 42. The distal end 38 of the proximal cage 32 and the proximal end 40 of the distal cage 34 are separated by an intervening portion or segment of tubing 44. A second cone 46, situated distal to the distal end 42 of distal cage 34, is not connected to the doublet filter cage assembly 26 and is fixed to the guidewire 22. The second cone 46 is also directed proximally (i.e., second cone 46 has a smaller diameter oriented proximally and a larger diameter oriented distally). A sheath 48 covers much of the guidewire 22 and has a distal end 50 which may be slid over, distally, or retracted from, proximally, the doublet filter cage assembly 26. When advanced fully (i.e., slid fully distally), the distal end 50 of the sheath 48 accepts the second cone 46. The proximal end of the sheath is not shown, but remains outside of a patient as does the proximal end of the guidewire 22. As shown in FIG. 1, the sheath 48 is retracted and the doublet filter cage assembly 26 including proximal cage 32 and distal cage 34 are both in an expanded state.

Preferably, the guidewire 22 is about 0.035 inch in diameter. Preferably, the sheath 48 has an outer diameter of about 0.092 inch and an inner diameter of about 0.082 inch. Such a sheath 48 corresponds to about # 6 or # 7 French scale. In an alternative, a # 6 French might be used in the sheath 48. Preferably, the sheath 48 is of a polyimide material, and most preferably, a braided polyimide material. Preferably, the proximal cone 30 has a length of about 0.320 inch and transistions from a proximal smaller end of about 0.035 inch to a distal end of about 0.072 inch. The proximal cone 30, if present, provides a smooth entrance of the doublet filter cage assembly 26 into the sheath 48 at distal end 50. Preferably, the proximal cone 30 is plastic or metal. Most preferably, the proximal cone 30, if plastic, is molded or bonded to the guidewire 22 and, if metal, is welded or crimped onto the guidewire 22. Preferably, the distal or second cone 46 has a length of about 0.320 inch and transistions from a distal smaller end of about 0.035 inch to a proximal end of about 0.072 inch, such that it may rest in distal end 50 of the sheath 48 when the sheath is fully advanced. Preferably, the cone 46 is plastic or metal. Most preferably, the distal free-floating cone 46, if plastic, is molded or bonded to the guidewire 22, and, if metal, is welded or crimped onto the guidewire 22. Such a cone 46 needs to be distally spaced to allow for distal expansion and contraction of the doublet filter cage assembly 26 and might alternatively be used to limit travel of the doublet filter cage assembly 26. Preferably, the shapeable tip 25 has a length of about 2.75 inches and extends distally from the second cone 46.

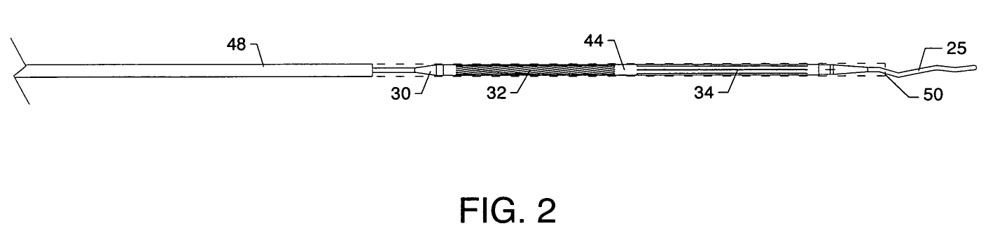

FIG. 2 shows portions of the sheath 48 in ghost or dotted outline so as to show the relationship of the doublet filter cage assembly 26, when collapsed, to the sheath 48. As shown in FIG. 2, when the sheath 48 is slid distally, the distal end 50 passes over the cone 30 and then sequentially causes the proximal cage 32 and the distal cage 34 to collapse. As they collapse, the proximal cage 32 and the distal cage 34 each increase in length while simultaneously decreasing in diameter. Upon completion of the distal movement of the sheath 48, both cages 32 and 34, as well as intervening portion or segment of tubing 44, are enclosed within the sheath 48. The distal end 50 of the sheath 48 then accepts the second cone 46. The shapeable tip 25 continues to project past the distal end 50 of the sheath 48 and is not enclosed by the sheath 48.

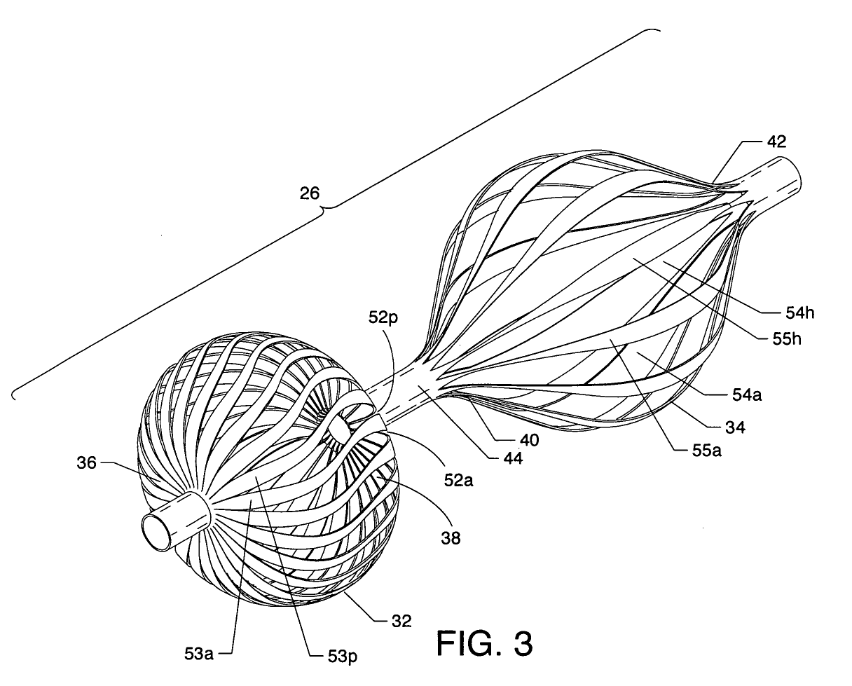

FIG. 3 shows the doublet filter cage assembly 26, in expanded state, independent of the guidewire 22. As previously pointed out, the doublet filter cage assembly 26 includes a proximal cage 32 with proximal end 36 and distal end 38, and a distal cage 24 with proximal end 40 and distal end 42. An intervening segment or portion of tube 44 separates the proximal cage 32 from the distal cage 34.

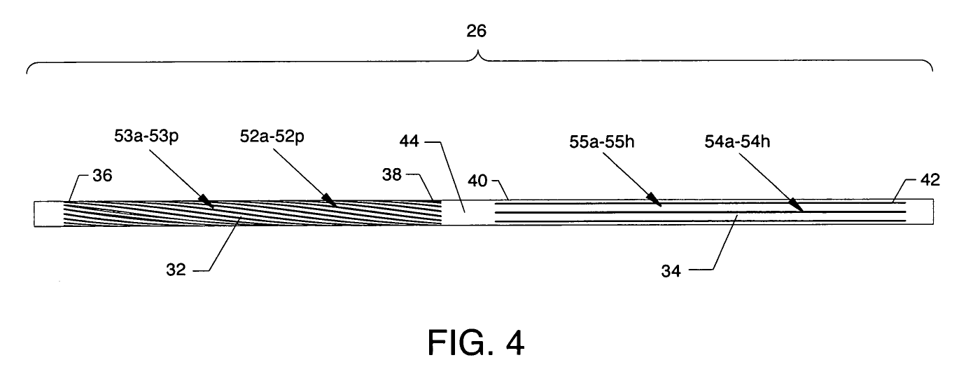

FIG. 4 shows the doublet filter cage assembly 26 independent of the guidewire 22 in a collapsed state. Proximal cage 32, in a collapsed state, is separated from distal cage 34 by intervening segment or portion of tubing 44. Also shown are helical cuts 52a-52p defining helical struts 53a-53p of proximal cage 32. Moreover, shown are straight cuts 54a-55h defining straight struts 55a-55h of distal cage 34. It should be recognized that the doublet cage filter assembly could be of a proximal helical filter cage and a distal longitudinal filter cage or, alternatively, a proximal longitudinal filter cage and a distal helical filter cage, or alternatively, two helical filter cages, or alternatively, two longitudinal filter cages.

Preferably, the doublet filter cage assembly 26 is prepared from nitinol tubing, especially nitinol tubing with an outer diameter of about 0.062 inch and an inner diameter of about 0.054 inch. Most preferably, the helical cuts 52a-52p and the straight cuts 54a-54h are about 0.003 inch in width and are radially directed on the nitinol tubing. Preferably, the helical cuts 52a-52p extend in a helical fashion and are regularly spaced apart from each other along the nitinol tubing for about 1.47 inches. Preferably, the straight cuts 54a-54h extend in a longitudinal fashion along the nitinol tubing for about 1.77 inches and are regularly spaced apart from each other. Cuts of such dimensions will result in cage filters 32 and 34 each having deployed or expanded dimensions of about 28 mm in diameter. Helical cuts 52a-52p of such dimensions, when expanded or deployed, will result in a cage filter 32 with a length of from about 10 mm to about 30 mm. Preferably, the helical angle of cuts 52a-52h is about 25 degrees. Most preferably, the helical cuts 52a-52p total 16 cuts and result in 16 helical struts 53a-53p. Most preferably, the longitudinal straight cuts 54a-54h total eight cuts and result in eight straight struts 55a-55h. Preferably, subsequent to forming the helical struts 53a-53p and straight struts 55a-55h by making helical cuts or slits 52a-52p and straight cuts or slits 54a-54h, respectively, the nitinol tubing is heat treated such that the expanded filter cages 32 and 34 (as shown in FIG. 3) resiliently attempt to assume the expanded or deployed state. One method to accomplish the heat treatment is to insert a sphere-like heat resistant object within the filter cages 32 and 34 and then exposed to appropriate heat for a sufficient time. An appropriate temperature would be below the annealing temperature for nitinol. A preferred heat resistant sphere-like object is a marble of diameter about 28 mm. Preferably, the heat is provided by a fluidized bed of sufficient temperature. It will be recognized that a variety of alternative objects could be used, as well as a variety of heat sources. Subsequently, the resulting resilient doublet filter cage assembly 26 is attached to the guidewire 22. In particular, the proximal end adjacent proximal end 36 of proximal filter cage 32 of doublet filter cage assembly 26 is attached to the guidewire by adhesive, solder, or welding. It should be understood that increasing the number of cuts, whether longitudinally or helically oriented, will increase the number of struts in a particular filter cage. A greater number of struts will increase filtration, but the struts tend to be less robust. A smaller number of struts will decrease filtration and allow larger particles to pass but will provide more robust struts and thereby a more robust filter cage.

In an alternative embodiment, instead of a sheath 48, actuation of the doublet filter cage assembly 26 may be generated by a mandrel design. For example, if the guidewire 22 were a tube, a mandrel may pass through the tube guidewire 22 to oppose and overcome the resilient nature of the doublet filter cage assembly 26. If the doublet filter cage assembly 26 resiliently is biased to the expanded state, the mandrel would force contraction by forcing the doublet filter cage assembly 26 to lengthen, or alternatively, if the doublet filter cage assembly 26 were biased to the contracted state, then the mandrel would force the doublet filter cage assembly 26 to shorten and thereby expand.

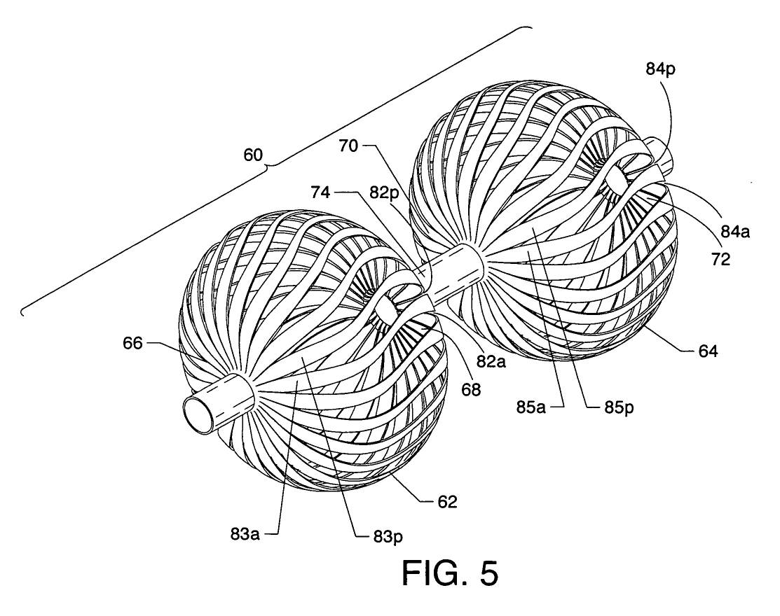

FIG. 5 shows an alternative embodiment doublet filter cage assembly 60. The alternative embodiment doublet filter cage assembly 60 includes a proximal filter cage 62 and a distal filter cage 64. The proximal filter cage 62 includes a proximal end 66 and a distal end 68 and the distal filter cage 64 includes a proximal end 70 and a distal end 72.

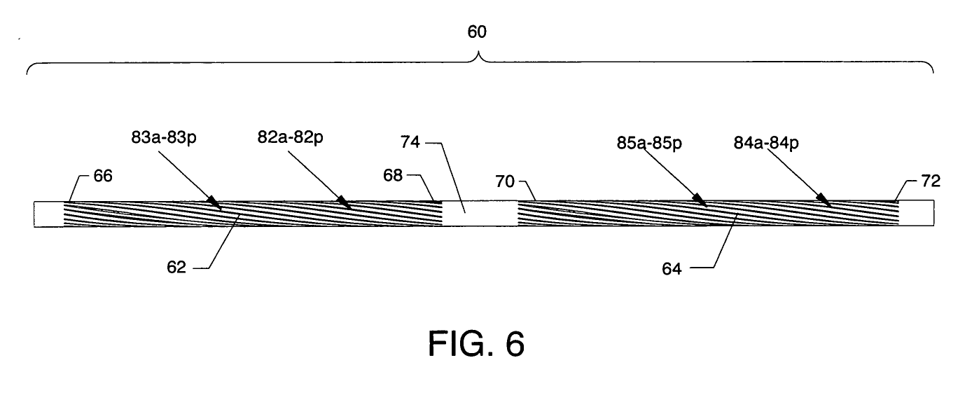

FIG. 6 shows the alternative embodiment doublet filter cage assembly 60 in an unexpanded or collapsed state, as would also be encountered during manufacture. A plurality of helical cuts 82a-82p define helical struts 83a-83p of proximal filter cage 62 and a second plurality of helical cuts 84a-84p define helical struts 85a-85p. An intervening tube portion 74 is present to separate the filter cages 62 and 64. The both pluralities of helical cuts 82a-82p and 84a-84p are radially oriented and radially distributed about the tubing and extend in helical fashion. Preferably, the tubing is nitinol, and preferably, the nitinol tubing is 0.062 outer diameter with 0.050 inner diameter tubing. Preferably, the helical cuts total 16 and are about 0.003 inch in width and arranged on about a 25 degree angle relative to the tubing axis. Preferably, both pluralities of cuts are identical. An alternative low profile cage filter may be made using a smaller nitinol tubing of 0.047 inch outer diameter and 0.038 inch inner diameter, along with the same pattern of two pluralities of 16 helical cuts. In both cases, the resulting expanded filter cages are about 28 mm in diameter and about 10 mm in longitudinal extent.

### MODE OF OPERATION

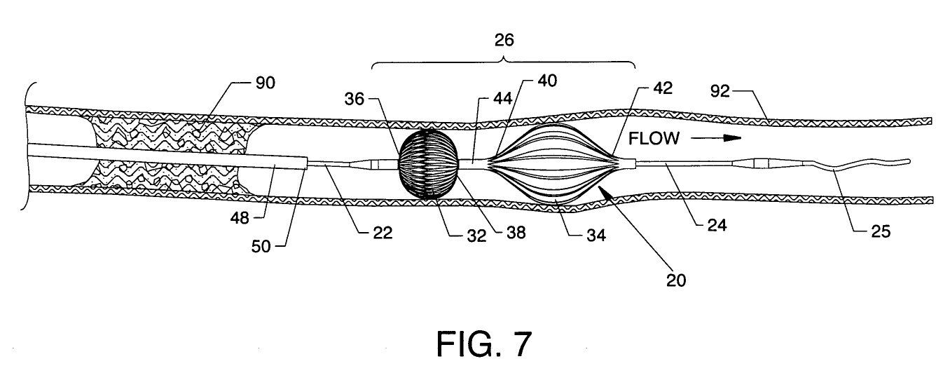

With reference to FIG. 7, two modes of operation of the present invention may be understood as follows. In both modes of operation, a physician initially evaluates and determines the location or site of the deep vein thrombosis 90. For example, consider a patient with deep vein thrombosis 90 in the right iliac vein 92 (leg vein). The physician may choose, in a first mode, to place the present invention 20 while accessing the right iliac vein 92 (i.e., push the device 20 through the thrombosis 90) and position the inferior vena cava doublet filter cage assembly 26 on guidewire 22 distal to the thrombus and thereby between the thrombus 90 and the patient's heart. In that first mode, the guidewire 22 could be used for delivering other interventional tools such as AngioJet® or infusion catheters or other thrombectomy devices. Alternatively, in a second mode of operation, the physician may decide to avoid crossing the thrombotic segment 90 with the present invention 20. In such second mode of operation, the physician may use a contralateral approach, accessing the deep vein thrombosis 90 through the left leg veins 92. In that case, the doublet cage filter assembly 26 of the present invention 20 could be positioned in the inferior vena cava. A separate guidewire (not shown) would be positioned across the thrombotic segment 90 for purposes of delivering interventional tools. As may be understood from these two modes of use, the present invention 20 enables a host of treatment options for the physician.

Once the selected interventional procedure is complete, the physician would use fluoroscopy to verify that the doublet filter cage assembly 26 of the device 20 was not occluded with thrombotic debris. If there was thrombotic debris occluding the doublet filter cage assembly 26, then a separate guidewire with AngioJet® could be delivered to the doublet filter cage assembly 26 and the doublet cage filter assembly 26 could be debulked prior to retrieval from the vein 92 of the patient. Retrieval is as simple as withdrawing the doublet filter cage assembly 26 back into sheath 48 via distal end 50 of sheath 48 and then withdrawing the device 20 from the vein 92 of the patient.

Alternatively, instead of debulking the doublet filter cage assembly 26 by using another separate guidewire and an AngioJet® or infusion catheter or other thrombectomy device, an AngioJet® or infusion catheter or other thrombectomy device might be directed to the doublet filter cage assembly 26 on the same guidewire 22 that is connected to the doublet filter cage assembly 26. In another variation, another additional inferior vena cava filter might be placed in the patient distal to the doublet filter cage assembly 26 by employing jugular access. This variation enables debris to be trapped by the additional inferior vena cava filter during removal of the doublet filter cage assembly 26. In yet another variation, it should be noted that collapsing the doublet filter cage assembly 26 tends to macerate any thrombus carried therein. The macerated thrombus would either be of such small particulate size as to be generally harmless or larger particulate sized macerated thrombus would be filtered out by the additional inferior vena cava filter, mentioned previously, or removed by an AngioJet® or infusion catheter or other thrombectomy device.

Various modifications can be made to the present invention without departing from the apparent scope thereof.

It is claimed:

1. A temporary inferior vena cava filter comprising:
a. a guidewire, the guidewire having a distal end and a proximal end; and,
b. a two-cage filter distally situated on the guidewire, the two-cage filter including:
(1) a first resilient filter cage, the first resilient filter cage actuatable between a collapsed state and a deployed state, and,
(2) a second resilient filter cage, the second resilient filter cage actuatable between a collapsed state and a deployed state, wherein the second resilient filter cage is situated distal to the first resilient filter cage.

2. The temporary inferior vena cava filter of claim 1, wherein the first resilient filter cage and the second resilient filter cage of the two-cage filter are actuated between the collapsed state and the deployed state by a sheath.

3. The temporary inferior vena cava filter of claim 1, wherein the first resilient filter cage and the second resilient filter cage of the two-cage filter are actuated between the collapsed state and the deployed state by a sheath of polyimide material.

4. The temporary inferior vena cava filter of claim 1, wherein the two-cage filter is a doublet filter cage assembly.

5. The temporary inferior vena cava filter of claim 4, wherein the doublet cage is formed from Nitinol tubing material.

6. The temporary inferior vena cava filter of claim 5, wherein the doublet cages are formed in the nitinol tubing by forming a first plurality of parallel cuts in the nitinol tubing at the desired location for one of the doublet cages and a second plurality of parallel cuts for the other of the doublet cages.

7. The temporary inferior vena cava filter of claim 6, wherein at least one of the plurality of parallel cuts are a plurality of helical cuts.

8. The temporary inferior vena cava filter of claim 6, wherein at least one of the plurality of parallel cuts are a plurality of linear, longitudinal, straight cuts.

9. The temporary inferior vena cava filter of claim 6, wherein the doublet cages are heat set in an expanded deployed state.

10. The temporary inferior vena cava filter of claim 5, wherein the proximal end of the nitinol tubing is fixed to the guidewire.

11. The temporary inferior vena cava filter of claim 2, wherein advancing the sheath forces the two-cage filter to a collapsed state and retracting the sheath allows the two-cage filter to resiliently expand to the expanded state.

12. The temporary inferior vena cava filter of claim 1, wherein the guidewire is about a 0.035 inch outer diameter guidewire.

13. The temporary inferior vena cava filter of claim 11, wherein the sheath is a braided polyimide sheath.

14. The temporary inferior vena cava filter of claim 11, wherein the sheath has an internal diameter of about 0.082 inch.

15. The temporary inferior vena cava filter of claim 1, further comprising a transitional cone between the guidewire and the doublet filter assembly.

16. The temporary inferior vena cava filter of claim 15, wherein the transitional cone is plastic or metal.

17. The temporary inferior vena cava filter of claim 5, wherein the nitinol tube is from about 0.062 inch to about 0.047 inch in outer diameter and from about 0.038 inch to about 0.054 inch in inner diameter.

18. The temporary inferior vena cava filter of claim 17, wherein there are from about eight to about 16 cuts in the pluralities of cuts in the nitinol tube.

19. The temporary inferior vena cava filter of claim 17, wherein the resilient cages have a diameter of about 28 mm.

20. A method of providing temporary protective filtering to protect from pulmonary embolism for a patient being treated for deep vein thrombosis, the method comprising the steps of:
a. providing a guidewire with a doublet cage filter, the doublet cage filter including at least two-cage filters actuatable between an expanded/deployable state and a collapsed/removable state and means for actuating the two-cage filters;
b. inserting the two-cage filters on the guidewire into a vein of the patient and advancing the two-cage filters to a protective location relative to the deep vein thrombosis and then deploying the two-cage filters at the protective location;
c. treating the deep vein thrombosis;
d. retracting the two-cage filters to the collapsed state; and,
e. withdrawing the two-cage filters in the collapsed state with the attached guidewire.

21. The method of claim 20, further comprising the step of:
f. debulking the two-cage filter prior to retraction.

22. The method of claim 20, wherein the step of advancing to a protective location relative to the deep vein thrombosis includes the step of:
g. passing through the deep vein thrombosis.

23. The method of claim 20, wherein the cage filters are resiliently expanded and wherein the guidewire with doublet cage filter further includes a sheath, which sheath may be advanced to collapse the cage filters or retracted to deploy and expand the cage filters and wherein step of deploying the cage filters includes retracting the sheath.

24. The method of claim 20, wherein the cage filters are resiliently expanded and wherein the guidewire with doublet cage filter further includes a sheath, which sheath may be advanced to collapse the cage filters or retracted to deploy and expand the cage filters and wherein step of retrieving the filters includes advancing the sheath to collapse the cage filters.

25. The method of claim 20, wherein the cage filters are formed of nitinol tubing material.

26. The method of claim 25, wherein the cage filters have from eight to 16 struts.

27. The method of claims 26, wherein at least one of the filter cages has struts that are linear when collapsed.

28. The method of claims 26, wherein at least one of the filter cages has struts that are helical when collapsed.

29. The method of claim 26, wherein the filter cages are about 28 mm in diameter in the expanded state.

30. A method of forming a filter cage assembly for attachment to the distal end of a guidewire, the method comprising the steps of:
a. providing a tube; and,
b. cutting a plurality of parallel slits in the tube to define a plurality of struts.

31. The method of claim 30, wherein the plurality of cuts are longitudinally oriented.

32. The method of claim 30, wherein the plurality of cuts are helically oriented.

33. The method of claim 30, wherein there are from eight to sixteen cuts in the plurality.

34. The method of claim 30, wherein the tube is nitinol and the cuts define a plurality of struts and the struts are heat treated in an expanded state.

35. The method of claim 34, wherein a heat resistant insert holds the struts in the expanded state during heat treatment.

36. The method of claim 34, further comprising the step of attaching to a guidewire.

37. The method of claim 36, further comprising the step of providing an actuator to force the struts into a retracted/collapsed state.

38. The method of claim 37, wherein the actuator is selected from a sheath or a mandrel.

39. The method of claim 30, wherein the filter cage is one of a plurality of filter cages on the same tube, each of the filter cages originating in a separate plurality of cuts to the tube.
